(19) United States
(12) Patent Application Publication
(10) Pub. No.: US 2023/0236161 A1
(43) Pub. Date: Jul. 27, 2023

(54) SEMICONDUCTOR METAL OXIDE BASED GAS SENSOR ACTIVATED AT ZERO HEATER POWER

(71) Applicants: STMicroelectronics PTE LTD, Singapore (SG); STMicroelectronics S.r.l., Agrate Brianza (MB) (IT)

(72) Inventors: Ravi SHANKAR, Singapore (SG); Wei Ren Douglas LEE, Toa Payoh (SG); Giuseppe BRUNO, Paterno' (IT)

(73) Assignees: STMicroelectronics PTE LTD, Singapore (SG); STMicroelectronics S.r.l., Agrate Brianza (MB) (IT)

(21) Appl. No.: 18/091,470

(22) Filed: Dec. 30, 2022

Related U.S. Application Data
(60) Provisional application No. 63/302,667, filed on Jan. 25, 2022.

(51) Int. Cl. G01N 33/00; G01N 27/04

(52) U.S. Cl. CPC G01N 33/0027; G01N 27/045

(57) ABSTRACT

A gas sensor is formed by a thin-film semiconductor metal-oxide gas sensing layer, with a thermally conductive and electrically-insulating layer in direct physical contact with a back of the gas sensing layer to carry the gas sensing layer. Sensing circuitry applies a voltage to the gas sensing layer and measures a current flowing through the gas sensing layer. The current flowing through the gas sensing layer is indicative of whether a gas under detection has been detected by the gas sensing layer, and serves to self-heat the gas sensing layer. A support structure extends from a substrate to make direct physical contact with and carry the thermally conductive and electrically insulating layer about a perimeter of a back face thereof, with the support structure shaped to form an air gap between the back of the thermally conductive and electrically insulating layer and a front of the substrate.

### RELATED APPLICATION

This application claims priority to United States Provisional Application for Patent No. 63/302,667, filed Jan. 25, 2022, the content of which is incorporated by reference in its entirety to the maximum extent allowable under the law.

### TECHNICAL FIELD

This disclosure is related to the field of semiconductor metal oxide-based gas sensors as used to test air for volatile organic compounds, with the gas sensors disclosed herein being designed to operate without the use of a heating element.

### BACKGROUND

Gas sensors formed using thin film metal oxide semiconductor layers are known and used in a variety of applications, such as testing indoor air quality in a closed environment like a room or building.

A common design for such a gas sensor 10 is shown in FIG. 1A. The gas sensor 10 includes a thin film metal oxide semiconductor layer 11 (gas sensing layer) with its front side exposed to the air to be tested, and with its back side in contact with a thermally conducting electrically insulating layer 12. A heater 13 is in contact with the thermally conducting electrically insulating layer 12. This gas sensor 10 performs resistance-type sensing, meaning that the resistance of the thin film metal oxide semiconductor layer 11 varies inversely proportional to the relative presence of the gas under detection at the front surface. In other words, there is a decrease in the film resistance corresponding to increased amount of the gas under detection, and conversely the resistance increases corresponding to a decrease in the amount of the gas under detection.

In greater detail, refer to the series of FIGS. 1B-1D. Shown in FIG. 1B is a tin(IV)-oxide (SnO₂) film, from which the gas sensing layer 11 may be formed. The SnO₂ film is multi-crystalline, formed of grains. At the grain boundaries, and at the surface, there will be molecules of SnO, which act as a reducing agent. At temperature (with heat being provided by the heater 13 and thermally conducted to the gas sensing layer 11 by the layer 12), atmospheric oxygen is adsorbed by the tin within the SnO molecules, as seen in FIG. 1C, to thereby form SnO₂ molecules, as seen in FIG. 1D. This has the effect of reducing free elections, as those electrons are used for forming bonds between the adsorbed oxygen atoms and the tin. Therefore, as heat is applied and oxygen is adsorbed, the conductance of the gas sensing layer 11 decreases, and the resistance of the gas sensing layer 11 increases.

If the gas under detection gas is present, the adsorbed gases on the surface of the gas sensing layer 11 reacts with the gas under detection. In FIG. 1D, the example of methane is shown, with the by-products of the reaction between the methane and the adsorbed gasses on the surface of the gas sensing layer 11 being carbon dioxide (CO₂), water (H₂O), and electrons that separate to thereby increase the conductivity of the gas sensing layer 11. Therefore, as stated, when the gas under detection is present in the vicinity of the gas sensor 10, the resistance of the gas sensing layer 11 decreases, with a greater drop in resistance of the layer 11 indicating a greater amount of detected gas.

The resistance of the gas sensing layer 11 is measured by applying a known voltage across the gas sensing layer 11, and then sensing the resulting current. The current will increase as the resistance of the gas sensing layer 11 decreases and will decrease as the resistance of the gas sensing layer 11 increases.

One technique for operating the heater 13 is described with additional reference to FIG. 1E, where it can be observed that outside of a measurement mode (a time when the gas to be detected is known to be present in the environment, such as during calibration of the gas sensor 10), the heater 13 is activated in pulses. During each pulse outside of measurement mode, the resistance of the gas sensing layer 11 increases (conductance decreases) due to the adsorption of atmospheric oxygen. During measurement mode, once a sufficient quantity of the gas to be detected is present and the heater 13 is activated, the resistance of the gas sensing layer 11 initially increases due to quick adsorption of atmospheric oxygen, but then reduces over time as the detected gas reacts with the adsorbed gases on the surface of the gas sensing layer 11.

While such prior art gas sensors 10 are effective for certain applications, one disadvantage lies in their usage of a heater, since operating the heater consumes power. Reducing power consumption is desirable. As such, further development is needed.

### SUMMARY

Disclosed herein is a gas sensor formed from a gas sensing layer formed by thin-film semiconductor metal oxide material, with a thermally conductive, electrically insulating layer being in direct physical contact with a back side of the gas sensing layer to thereby support the gas sensing layer. Sensing circuitry is configured to apply a voltage to the gas sensing layer and measure a resulting current flowing through the gas sensing layer. The current flowing through the gas sensing layer is indicative of whether a gas under detection has been detected by the gas sensing layer, and serves to self-heat the gas sensing layer. A support structure makes direct physical contact with and carry the thermally conductive, electrically insulating layer about a perimeter of a back face thereof, with the support structure being shaped such that an air gap is formed between the back face of the thermally conductive and electrically insulating layer and the support structure.

Electrical conductance of the gas sensing layer increases in the presence of the gas under detection and decreases in the absence of the gas under detection.

The thin-film semiconductor metal oxide material may be formed from tin(IV)-oxide (SnO₂), tungsten(III)-oxide (W₂O₃), and/or zinc oxide (ZnO).

The thin-film semiconductor metal oxide material may include a dopant, such as platinum or palladium.

The gas sensing layer may have a thickness of between 50 nm and 60 nm.

The support structure may extend from a substrate, and the air gap may be formed between the back face of the thermally conductive, electrically insulating layer and a front face of the substrate.

Also disclosed herein is a method, including applying a voltage to a gas sensing layer carried by a thermally conductive, electrically insulating layer that itself is carried by a support structure, and self-heating the gas sensing layer using current flowing through the gas sensing layer as a result of the applied voltage, also resulting in heating of air within an air gap defined between the support structure and the thermally conductive, electrically insulating layer. The method further includes detecting a gas under detection by measuring the current flowing through the gas sensing layer, with an increase in the current indicating presence of the gas under detection and a decrease in the current indicating an absence of the gas under detection.

The method may also include forming the gas sensing layer from a thin-film semiconductor metal oxide material, with the thin-film semiconductor metal oxide material being at least one of tin(IV)-oxide (SnO₂), tungsten(III)-oxide (W₂O₃), and zinc oxide (ZnO).

The method may also include doping the thin-film semiconductor metal oxide material with a dopant, the dopant being at least one of platinum, and palladium.

The gas sensing layer may be formed to have a thickness of between 50 nm and 60 nm.

Also disclosed herein is a gas sensor, including a gas sensing layer, a thermally conductive, electrically insulating layer in direct physical contact with a back side of the gas sensing layer to thereby support the gas sensing layer, sensing circuitry configured to determine whether a gas under detection has been detected by the gas sensing layer while causing self-heating of the gas sensing layer, and a support structure carrying the thermally conductive, electrically insulating layer about a perimeter of a back face thereof, with the support structure being shaped such that an air gap is formed between the back face of the thermally conductive and electrically insulating layer and the support structure.

An electrical conductance of the gas sensing layer may increase in the presence of the gas under detection and decreases in the absence of the gas under detection.

The gas sensing layer may have thickness of between 50 nm and 60 nm.

The gas sensor may include a substrate from which the support structure extends, and the air gap may be formed between the back face of the thermally conductive, insulating layer and a front face of the substrate.

The gas sensing layer may be constructed from a doped thin-film semiconductor metal oxide material.

The doped thin-film semiconductor metal oxide material may be formed from at least one of tin(IV)-oxide (SnO₂), tungsten(III)-oxide (W₂O₃), and zinc oxide (ZnO).

The doped thin-film semiconductor metal oxide material may be doped with at least one of platinum, and palladium.

### BRIEF DESCRIPTION OF THE DRAWINGS

FIG. 1A is a cross sectional view of a known prior art gas sensor.

FIG. 1B diagrammatically depicts the grain structure of the gas sensing layer of the prior art gas sensor of FIG. 1A.

FIG. 1C diagrammatically depicts adsorption of atmospheric oxygen at the surface of the gas sensing layer of the prior art gas sensor of FIG. 1A.

FIG. 1D diagrammatically depicts the reaction between a gas under detection and the surface of the gas sensing layer.

FIG. 1E is a graph illustrating the response of the prior art gas sensor of FIG. 1 in the presence of a gas under detection.

FIG. 2 is a cross sectional view of a gas sensor disclosed herein.

FIGS. 3A-3C diagrammatically illustrates the process of self-heating of the gas sensing layer of the gas sensor of FIG. 2, and the process of the sensing of a gas under detection by the gas sensing layer of the gas sensor of FIG. 2.

FIGS. 4A-4C diagrammatically illustrates the process of self-heating of the gas sensing layer of the gas sensor of FIG. 2 when the gas sensing layer including a dopant, and the process of the sensing of a gas under detection by that gas sensing layer.

FIG. 5 is a graph showing the response of the gas sensor of FIG. 2 when exposed to a gas containing volatile organic compounds.

FIG. 6 is a cross sectional view of an alternative embodiment of a gas sensor disclosed herein.

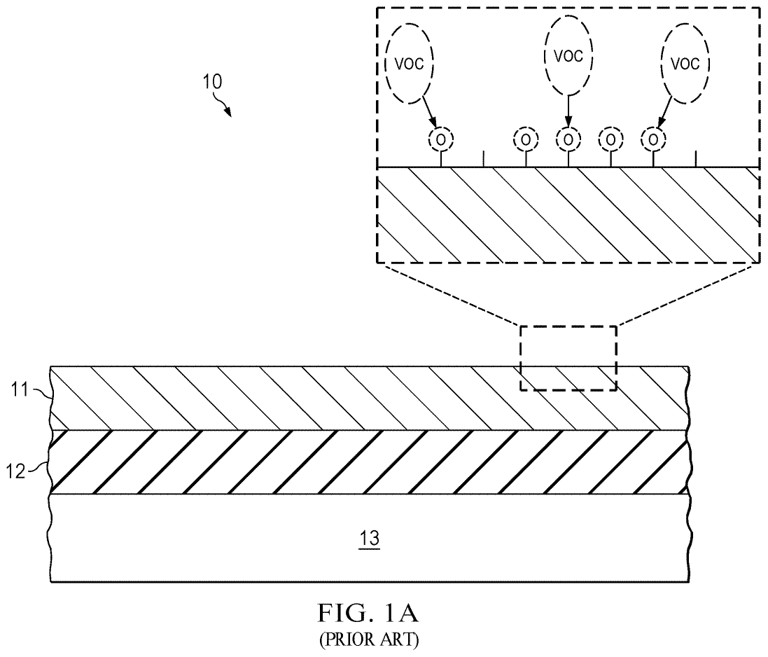

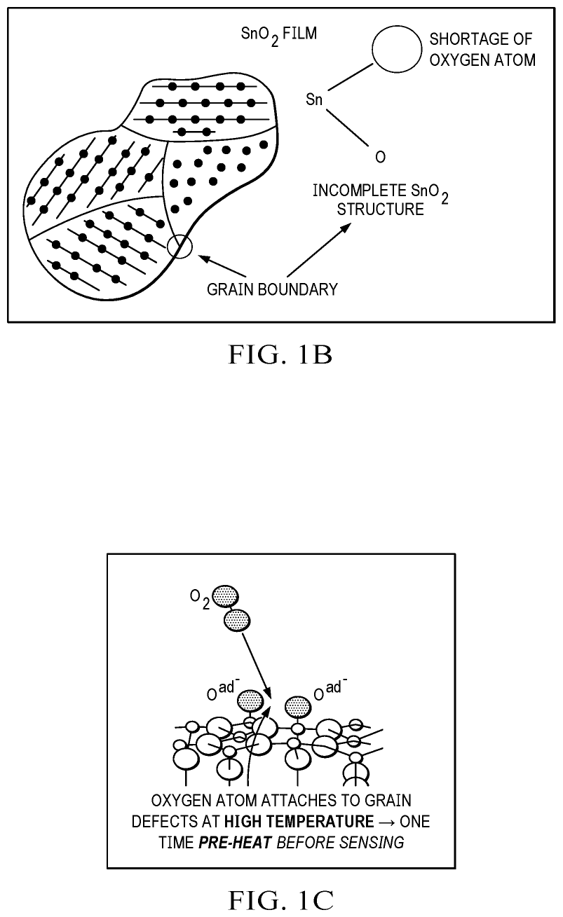

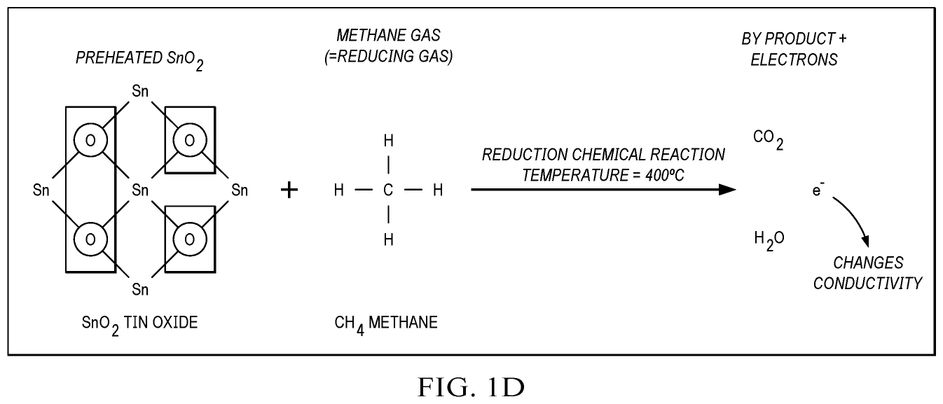

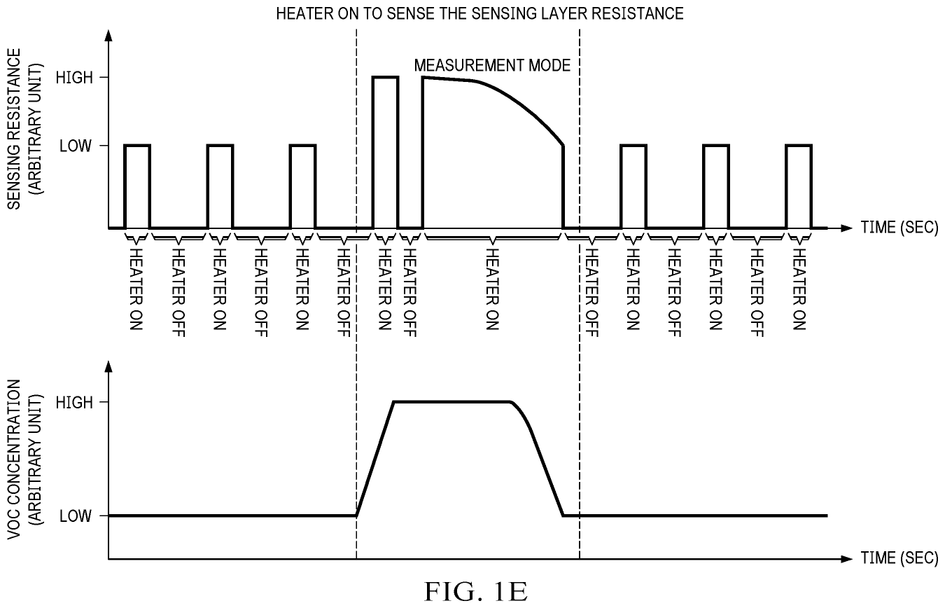

### DETAILED DESCRIPTION

The following disclosure enables a person skilled in the art to make and use the subject matter disclosed herein. The general principles described herein may be applied to embodiments and applications other than those detailed above without departing from the spirit and scope of this disclosure. This disclosure is not intended to be limited to the embodiments shown, but is to be accorded the widest scope consistent with the principles and features disclosed or suggested herein. Do note that in the below description, any described resistor or resistance is a discrete device unless the contrary is stated, and is not simply an electrical lead between two points. Thus, any described resistor or resistance coupled between two points has a greater resistance than a lead between those two points would have, and such resistor or resistance cannot be interpreted to be a lead. Similarly, any described capacitor or capacitance is a discrete device unless the contrary is stated, and is not a parasitic unless the contrary is stated. Moreover, any described inductor or inductance is a discrete device unless the contrary is stated, and is not a parasitic unless the contrary is stated.

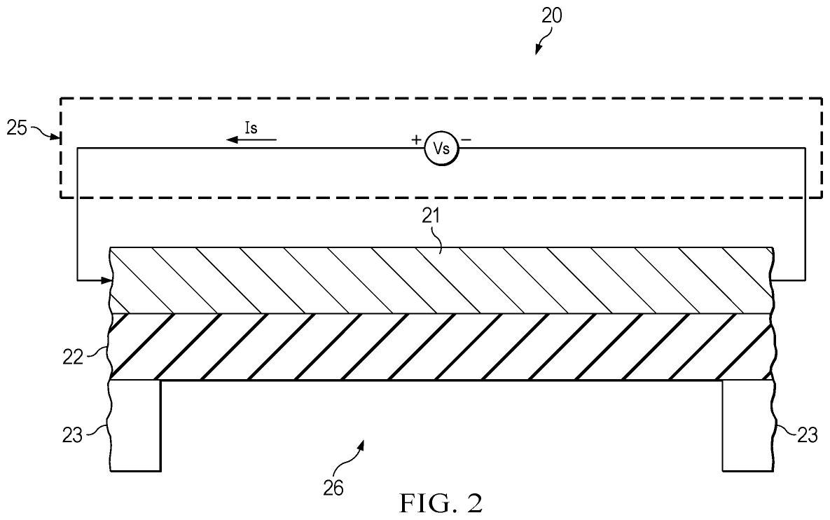

A gas sensor 20 is now described with initial reference to FIG. 2, and may be used to detect volatile organic compounds (VOC), such as ethanol, formaldehyde, toluene, benzene, etc. The gas sensor 20 includes a thin film metal oxide semiconductor layer 21 (gas sensing layer) which may be, for example, tin(IV)-oxide (SnO₂) film, tungsten(III)-oxide (W₂O₃) film, zinc oxide (ZnO) film, etc., having a thickness on the order of 50 nm-60 nm. The front side of the gas sensing layer 21 is exposed to the air to be tested, and the back side of the gas sensing layer 21 is in contact with a thermally conducting electrically insulating layer 22 (e.g., silicon nitride, silicon oxynitride, silicon dioxide, etc). Supports 23 carry the thermally conducting electrically insulating layer 22, creating an air gap 26 under the back side of the thermally conducting electrically insulating layer 22.

The gas sensor 20 performs resistance-type sensing, with the resistance of the gas sensing layer 21 decreasing if the gas under detection is present in the vicinity of the gas sensor 20, and the resistance of the gas sensing layer 21 increasing if the gas under detection does not exist within the vicinity of the gas sensor 20. Sensing circuitry 25 applies a known sensing voltage Vs across the gas sensing layer 21, and measures the current Is flowing through the gas sensing layer 21 as a result. The higher the current, the lower the resistance of the gas sensing layer 21 and therefore the greater the amount of detected gas.

Notice that there is no heater or heating layer between the thermally conducting electrically insulating layer 22 and the air gap 26, and in fact that the gas sensor 20 lacks a heater or heating layer, with the heat in the gas sensor 20 being provided solely by power dissipation within the gas sensing layer 21.

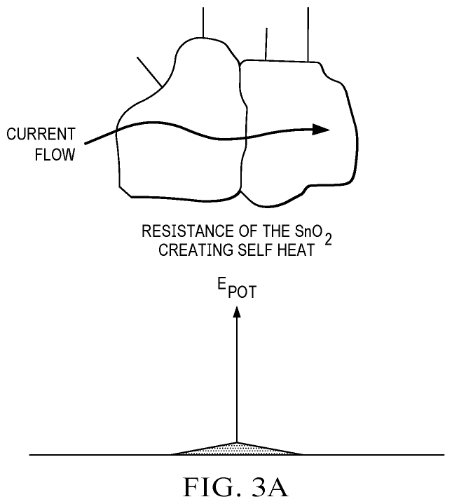

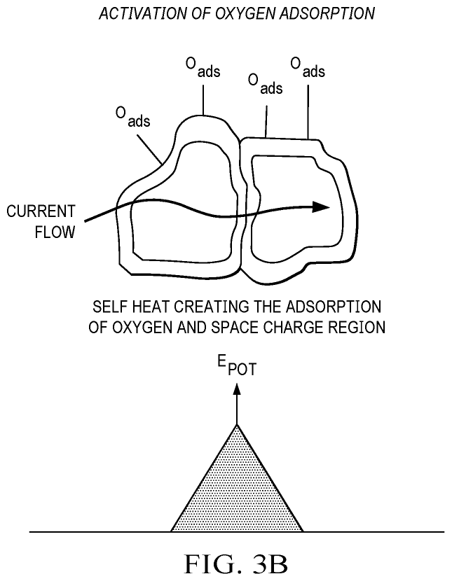

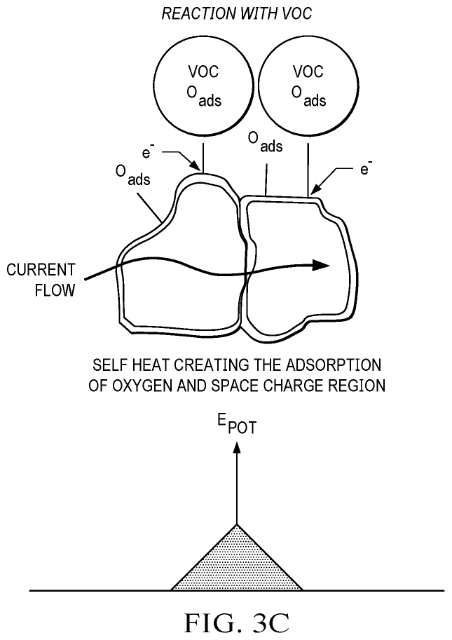

As a result of the application of the sensing voltage Vs, current flows through the gas sensing layer 21, and because the layer 21 is resistive there is some energy is dissipated in the form of heat. Shown in FIG. 3A are two grains of the multi-crystalline structure of the gas sensing layer 21 and the current flow thereacross during a start-up condition. Also shown is that a gradient in potential across the grain barrier is small.

As the heat generated by the current flow begins to heat up the gas sensing layer 21, atmospheric oxygen begins to be adsorbed by the grains, as shown in FIG. 3B. As a result of the adsorbed oxygen being present, a space charge region forms at the grain barrier, and therefore the gradient in potential across the grain barrier increases.

When a gas under detection (e.g., a VOC) is in the vicinity of the gas sensor 20, the gas under detection reacts with the adsorbed gases on the surface of the gas sensing layer 21 yielding, as one of the products, free electrons that decrease the space charge region and lower the potential gradient across the grain barrier to thereby increase the conductance (increase the resistance) of the gas sensing layer 21, as shown in FIG. 3C.

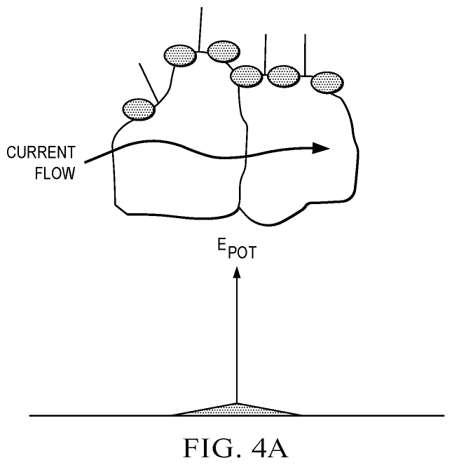

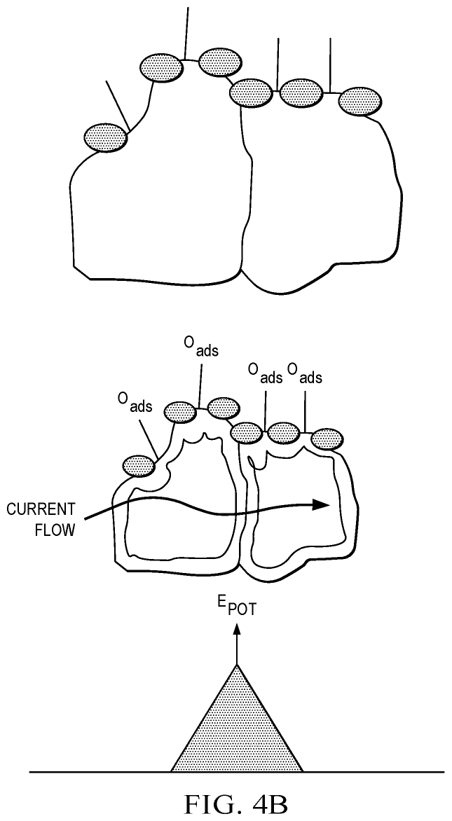

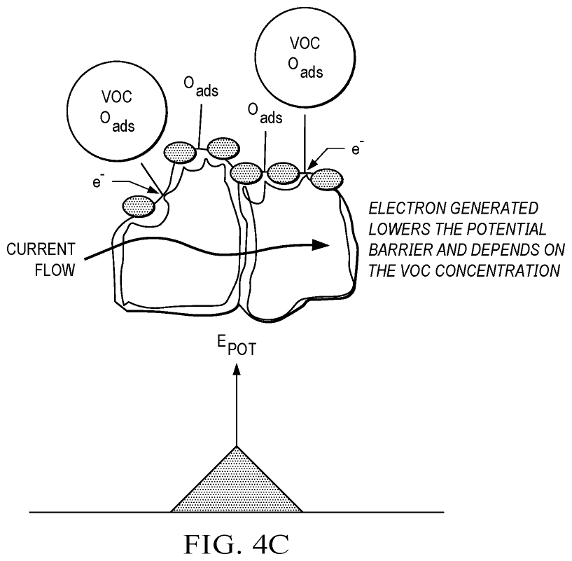

The gas sensing layer 21 may be doped with a catalyst (e.g., platinum or palladium), which has the effect of further lowering the gradient in potential across the grain barrier due to self heating, reducing power consumption, as may be observed in FIGS. 4A-4C.

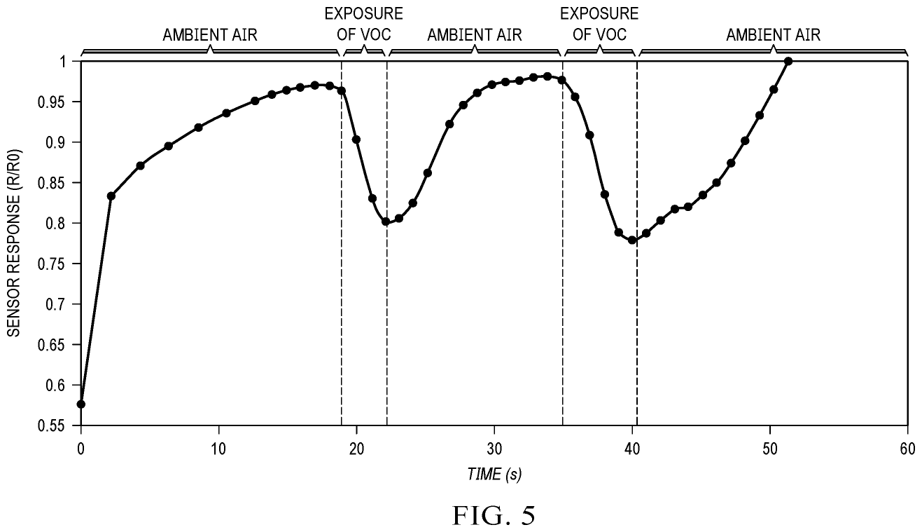

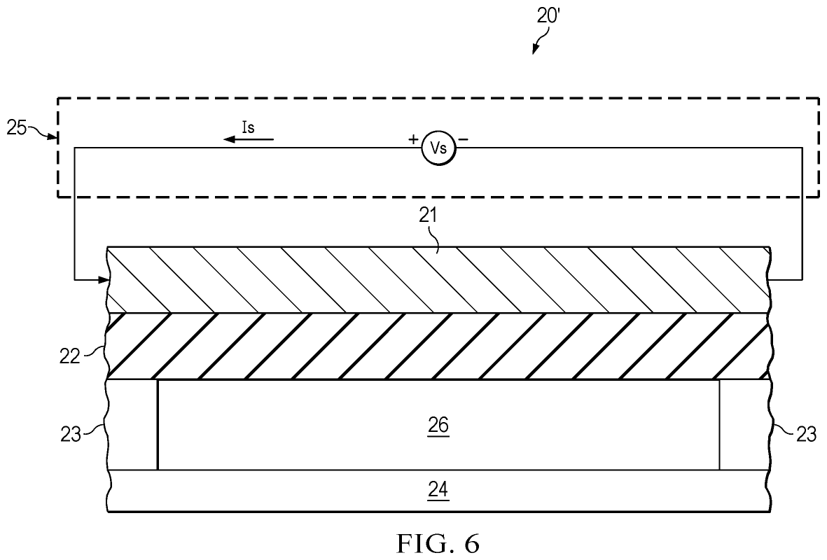

The response of the gas sensing layer 21 (changing in resistance) may be seen in the graph of FIG. 5, where it can be observed that the resistance initially increases upon start-up as atmospheric oxygen is adsorbed by the gas sensing layer 21, but once the gas sensing layer 21 is exposed to a gas under detection such as a VOC, the resistance of the gas sensing layer 21 sharply drops due to the reaction between the adsorbed atmospheric oxygen and the gas under detection. As also shown, once the supply of the VOC is removed and fresh air is supplied, the resistance of the gas sensing layer 21 again increased, and then once a VOC is reintroduced, the resistance of the gas sensing layer 21 again sharply drops, illustrating the repeatability of the operation of the gas sensor 20.

The benefits of this gas sensor design 20 include power savings, since there is no heat source other than the gas sensing layer 21 consuming power, as well as a quick response time due to the thinness of the gas sensing layer 21, and use of the air gap 26 to heat and trap air to keep as much of the heat generated by the gas sensing layer 21 from being radiated other than to the thermally conducting, electrically insulating layer 22 which helps evenly apply the heat it absorbs to the gas sensing layer 21.

Finally, it is clear that modifications and variations may be made to what has been described and illustrated herein, without thereby departing from the scope of this disclosure, as defined in the annexed claims.

While the disclosure has been described with respect to a limited number of embodiments, those skilled in the art, having benefit of this disclosure, will appreciate that other embodiments can be envisioned that do not depart from the scope of the disclosure as disclosed herein. As an example, the supports 23 may extend from a substrate 24 to carry the thermally conducting electrically insulating layer 22, creating the air gap 26 as between the thermally conducting electrically insulating layer 22 and the substrate 24. Accordingly, the scope of the disclosure shall be limited only by the attached claims.

1. A gas sensor, comprising:
a gas sensing layer made of a thin-film semiconductor metal oxide material;
a thermally conductive and electrically insulating layer in direct physical contact with a back side of the gas sensing layer to thereby support the gas sensing layer;
sensing circuitry configured to apply a voltage to the gas sensing layer and measure a resulting current flowing through the gas sensing layer;
wherein the current flowing through the gas sensing layer is indicative of whether a gas under detection has been detected by the gas sensing layer, and serves to self-heat the gas sensing layer; and
a support structure making direct physical contact with and carrying the thermally conductive and electrically insulating layer about a perimeter of a back face thereof, with the support structure being shaped such that an air gap is formed between the back face of the thermally conductive and electrically insulating layer and the support structure to thereby trap air and heat generated by the gas sensing layer from being radiated other than to the thermally conductive and electrically insulating layer.

2. The gas sensor of claim 1, wherein an electrical conductance of the gas sensing layer increases in the presence of the gas under detection and decreases in the absence of the gas under detection.

3. The gas sensor of claim 1, wherein the thin-film semiconductor metal oxide material comprises at least one of tin(IV)-oxide (SnO₂), tungsten(III)-oxide (W₂O₃), and zinc oxide (ZnO).

4. The gas sensor of claim 1, wherein the thin-film semiconductor metal oxide material includes a dopant.

5. The gas sensor of claim 4, wherein the dopant comprises at least one of platinum and palladium.

6. The gas sensor of claim 1, wherein the gas sensing layer has a thickness of between 50 nm and 60 nm.

7. The gas sensor of claim 1, further comprising a substrate from which the support structure extends, and wherein the air gap is formed between the back face of the thermally conductive, insulating layer and a front face of the substrate.

8. A method, comprising:
applying a voltage to a gas sensing layer carried by a thermally conductive and electrically insulating layer that itself is carried by a support structure;
self-heating the gas sensing layer using current flowing through the gas sensing layer as a result of the applied voltage, also resulting in heating of air within an air gap defined between the support structure and the thermally conductive and electrically insulating layer; and
detecting a gas under detection by measuring the current flowing through the gas sensing layer, with an increase in the current indicating presence of the gas under detection and a decrease in the current indicating an absence of the gas under detection.

9. The method of claim 8, further comprising forming the gas sensing layer from a thin-film semiconductor metal oxide material.

10. The method of claim 9, wherein the thin-film semiconductor metal oxide material comprises at least one of tin(IV)-oxide (SnO₂), tungsten(III)-oxide (W₂O₃), and zinc oxide (ZnO).

11. The method of claim 9, further comprising doping the thin-film semiconductor metal oxide material with a dopant.

12. The method of claim 11, wherein the dopant comprises at least one of platinum, and palladium.

13. The method of claim 8, wherein the gas sensing layer is formed to have a thickness of between 50 nm and 60 nm.

14. A gas sensor, comprising:
a gas sensing layer;
a thermally conductive and electrically insulating layer in direct physical contact with a back side of the gas sensing layer to thereby support the gas sensing layer;
sensing circuitry configured to determine whether a gas under detection has been detected by the gas sensing layer while causing self-heating of the gas sensing layer; and
a support structure carrying the thermally conductive and electrically insulating layer about a perimeter of a back face thereof, with the support structure being shaped such that an air gap is formed between the back face of the thermally conductive and electrically insulating layer and the support structure.

15. The gas sensor of claim 14, wherein an electrical conductance of the gas sensing layer increases in the presence of the gas under detection and decreases in the absence of the gas under detection.

16. The gas sensor of claim 14, wherein the gas sensing layer has a thickness of between 50 nm and 60 nm.

17. The gas sensor of claim 14, further comprising a substrate from which the support structure extends, and wherein the air gap is formed between the back face of the thermally conductive, insulating layer and a front face of the substrate.

18. The gas sensor of claim 14, wherein the gas sensing layer is constructed from a doped thin-film semiconductor metal oxide material.

19. The gas sensor of clam 18, wherein the doped thin-film semiconductor metal oxide material comprises at least one of tin(IV)-oxide (SnO₂), tungsten(III)-oxide (W₂O₃), and zinc oxide (ZnO).

20. The gas sensor of clam 18, wherein the doped thin-film semiconductor metal oxide material is doped with at least one of platinum, and palladium.
